(19) United States
(12) Patent Application Publication
(10) Pub. No.: US 2010/0160454 A1
(43) Pub. Date: Jun. 24, 2010

(54) ANTIMICROBIAL AGENTS, COMPOSITIONS AND PRODUCTS CONTAINING THE SAME, AND METHODS OF USING THE COMPOSITIONS AND PRODUCTS

(75) Inventors: James Allen McCaulley, Ringoes, NJ (US); Terry Ann Oldfield, Kingsport, TN (US); Andrew Joseph Matosky, Kingsport, TN (US); Suzanne Winegar Dobbs, Kingsport, TN (US); Vicky Lynn Christian, Mount Carmel, TN (US); Thelma Lee Watterson, Kingsport, TN (US); Kab Sik Seo, Kingsport, TN (US); Nancy Kinkade, Kingsport, TN (US); Jessica Posey-Dowty, Kingsport, TN (US)

Correspondence Address: BRETT L. NELSON;EASTMAN CHEMICAL COMPANY, 100 NORTH EASTMAN ROAD, KINGSPORT, TN, 37660-5075, US

(73) Assignee: Eastman Chemical Company, Kingsport, TN (US)

(21) Appl. No.: 12/615,639

(22) Filed: Nov. 10, 2009

Related U.S. Application Data
(62) Division of application No. 12/341,462, filed on Dec. 22, 2008.

(51) Int. Cl. A01N 31/00; A61K 31/045; A01P 1/00; A61P 31/00

(52) U.S. Cl. 514/729

(57) ABSTRACT

Antimicrobial agents, products and compositions incorporating the agents, and methods of using the compositions and products are provided. The antimicrobial agents comprise 1,2-cyclohexanedimethanol, 1,4-cyclohexanedimethanol, 2,2,4,4-tetramethyl-1,3-cyclobutanediol, or mixtures thereof. These agents have surprisingly been found to inhibit microbial growth at a much lower concentration than other glycols with known antimicrobial activity.

### CROSS-REFERENCE TO RELATED APPLICATION

This application is a divisional of U.S. patent application Ser. No. 12/341,462, filed on Dec. 22, 2008, the disclosure of which is incorporated herein by reference in its entirety.

### FIELD OF THE INVENTION

The invention generally pertains to antimicrobial agents, compositions and products incorporating the agents, and methods of using the compositions and products. The antimicrobial agents are 1,2-cyclohexanedimethanol, 1,4-cyclohexanedimethanol, 2,2,4,4-tetramethyl-1,3-cyclobutanediol, and mixtures thereof.

### BACKGROUND OF THE INVENTION

Many compositions and products, including personal care, medicinal, animal care, household care, fuel, and oil, often contain water or can accumulate water from the environment. Water makes the compositions and products susceptible to microbial growth.

Preservatives are typically added to these products to limit the growth of any bacteria, yeast, or mold. Many different types of preservatives are available for this purpose. The type of preservative and their concentration are selected based on a number of factors including the type of product being preserved, the efficacy of the preservative, and the types of organisms that are likely to contaminate the product. If the product is likely to come into contact with humans or animals, the preservative has to be considered for potential for causing irritation, dryness, allergy, and toxicity. Due to these and other considerations, government institutions sometimes regulate the use of preservatives.

The number of effective preservatives that can be used is becoming more limited, not only because of government regulation, but also because of consumer concern about their potential for harm to the consumer or the environment.

Many glycols have been identified as having preservative effect such that traditional preservatives can be eliminated from the products or their concentration can be reduced. Such glycols include propylene glycol, butylene glycol, pentylene glycol, 1,2-hexanediol, 1,2-octanediol, 1,5-pentanediol, methyl propanediol, and 1,3-alkanediols having 5 to 15 carbon atoms. The 1,2-hexanediol and 1,2-octanediol have been found to be particularly effective as antibacterial agents, and it has been recognized that the antibacterial activity of 1,2-alkanediols increases as the alkyl chain length increases. The hydrophobic interaction of the longer hydrocarbon chain with microorganisms is thought to contribute to their antibacterial activity. However, as the alkyl chain length increases, the water solubility of these compounds decreases. For certain products containing an immiscible organic phase (such as personal care emulsions), compounds having low water solubility are likely to migrate into the oil phase where they are less effective.

Thus, there is a continuing need in the art for antimicrobial agents that are effective, preferably at lower concentrations; that are safe; that cause minimal allergic reaction, irritation, and dryness at the effective concentrations; and that have a high degree of solubility in water at ambient or near ambient conditions.

### SUMMARY OF THE INVENTION

It has been surprisingly found that 1,4-cyclohexanedimethanol (1,4-CHDM) and its isomers 1,2-cyclohexanedimethanol (1,2-CHDM) and 2,2,4,4-tetramethyl-1,3-cyclobutanediol (TMCBD) (collectively CHDM) have antimicrobial activity and that they can inhibit microbial growth at much lower concentrations than other glycols with known antimicrobial activity. CHDM also has a greater solubility in water compared to other glycols of similar molecular weight.

In a first aspect, the invention provides a method for reducing or inhibiting microbial growth in an aqueous composition. The method comprises adding an antimicrobial agent selected from the group consisting of 1,2-cyclohexanedimethanol, 1,4-cyclohexanedimethanol, and 2,2,4,4-tetramethyl-1,3-cyclobutanediol to the composition.

In a second aspect, the invention provides a composition comprising (a) a fuel or oil selected from diesel, biodiesel, a mixture of diesel and biodiesel, aviation fuel, hydraulic oil, lubrication oil, vegetable oil, crude oil, transmission fluid, heating oil, or kerosene; and (b) an antimicrobial agent selected from the group consisting of 1,2-cyclohexanedimethanol, 1,4-cyclohexanedimethanol, and 2,2,4,4-tetramethyl-1,3-cyclobutanediol.

In a third aspect, the invention provides a personal care product comprising about 1 to 5 weight percent of an antimicrobial agent selected from the group consisting of 1,2-cyclohexanedimethanol, 1,4-cyclohexanedimethanol, and 2,2,4,4-tetramethyl-1,3-cyclobutanediol.

In a fourth aspect, the invention provides a medicated product comprising a medicinal substance; and about 1 to 5 weight percent of an antimicrobial agent selected from the group consisting of 1,2-cyclohexanedimethanol, 1,4-cyclohexanedimethanol, and 2,2,4,4-tetramethyl-1,3-cyclobutanediol.

In a fifth aspect, the invention provides an animal care product comprising about 1 to 5 weight percent of an antimicrobial agent selected from the group consisting of 1,2-cyclohexanedimethanol, 1,4-cyclohexanedimethanol, and 2,2,4,4-tetramethyl-1,3-cyclobutanediol.

In a sixth aspect, the invention provides a household care product comprising about 1 to 5 weight percent of an antimicrobial agent selected from the group consisting of 1,2-cyclohexanedimethanol, 1,4-cyclohexanedimethanol, and 2,2,4,4-tetramethyl-1,3-cyclobutanediol.

In a seventh aspect, the invention provides a method for providing residual antimicrobial activity to a surface. The method comprises topically applying the personal care, medicated, animal care, or household care product mentioned above to the surface, and optionally removing any excess amounts of the product from the surface.

In an eighth aspect, the invention provides a method for preventing or reducing odor from the presence of bacteria or fungi on a mammalian surface. The method comprises topically applying the personal care, medicated, or animal care product mentioned above to the mammalian surface, and optionally removing any excess amounts of the product from the mammalian surface.

In a ninth aspect, the invention provides a method for providing antimicrobial activity to a film, fiber, molded or extruded article, or composite material made of fibers, polymers, adhesives, and/or gypsum. The method comprises incorporating an antimicrobial agent selected from the group consisting of 1,2-cyclohexanedimethanol, 1,4-cyclohexanedimethanol, and 2,2,4,4-tetramethyl-1,3-cyclobutanediol into the film, fiber, molded or extruded article, or composite material during its manufacturing process.

### DETAILED DESCRIPTION OF THE INVENTION

According to a first aspect, the invention provides a method for reducing or inhibiting microbial growth in an aqueous composition. The method comprises adding an antimicrobial agent selected from the group consisting of 1,2-cyclohexanedimethanol, 1,4-cyclohexanedimethanol, and 2,2,4,4-tetramethyl-1,3-cyclobutanediol to the aqueous composition.

In one embodiment, 1,2-CHDM, 1,4-CHDM, TMCBD, or a mixture thereof is the only antimicrobial agent in the composition.

The aqueous composition can be any composition that contains water and that is susceptible to microbial growth. Examples of such compositions include fuel or oil compositions, personal care products, medicated products, animal care products, and household care products. Thus, in addition to water, the aqueous composition can contain, for example, an organic compound such as hydrocarbons, triglycerides, fatty acids, fatty acid alkyl esters, fatty alcohols, polyglycol ethers, alkyl glycol ethers, alkyl glycol esters, alkyl glycol ether esters, alkyl amines, alkyl amides, and mixtures thereof. Other examples of the organic compound include diesel, biodiesel, a mixture of diesel and biodiesel, aviation fuel, hydraulic oil, lubrication oil, vegetable oil, crude oil, transmission fluid, heating oil, or kerosene.

In one embodiment, the organic compound and the water in the aqueous composition are miscible. In another embodiment, the organic compound and the water in the aqueous composition are in separate liquid phases. In this latter case, the antimicrobial agent preferably reduces or inhibits microbial growth at the interface between the organic phase and the aqueous phase in the aqueous composition.

The amount of the antimicrobial agent present in the aqueous composition can vary depending on various factors including the application of the aqueous composition and the degree of microbial protection desired. Generally, the antimicrobial agent can be present in an amount of about 1 to 5 weight percent, based on the total weight of the composition. The agent can also be present in an amount of about 1 to 3 weight percent, based on the total weight of the composition.

The manner in which the antimicrobial agent is added to the aqueous composition is not particularly limiting. For example, the antimicrobial agent may be added to the aqueous composition by simply combining the agent with the composition and mixing the ingredients. Alternatively, the antimicrobial agent, due to its high solubilizing power, may be used as a solvent for one or more of the ingredients of the aqueous composition before it is mixed with the remainder of the composition ingredients.

In another embodiment, the antimicrobial agent may be added to the aqueous composition by first mixing the agent with a solvent that is immiscible with water and then combining the agent-solvent mixture with the aqueous composition.

The antimicrobial agent itself may be a soft solid at room temperature. Therefore, to facilitate mixing and/or handling, the agent may first be diluted with up to 10 wt % or more of water before it is combined with the aqueous composition or the ingredients thereof.

The method of the invention is effective to reduce or inhibit microbial growth of various kinds including biofilms.

According to a second aspect, the invention provides a composition comprising (a) a fuel or oil selected from diesel, biodiesel, a mixture of diesel and biodiesel, aviation fuel, hydraulic oil, lubrication oil, vegetable oil, crude oil, transmission fluid, heating oil, or kerosene; and (b) an antimicrobial agent selected from the group consisting of 1,2-cyclohexanedimethanol, 1,4-cyclohexanedimethanol, and 2,2,4,4-tetramethyl-1,3-cyclobutanediol.

The amount of the antimicrobial agent present in the fuel or oil composition can vary depending on various factors including the degree of microbial protection desired. Generally, the antimicrobial agent can be present in an amount of about 0.01 to 1 weight percent, based on the total weight of the composition. The agent can also be present in an amount of about 0.02 to 0.5 weight percent, based on the total weight of the compositionor even in an amount of about 0.05 to 0.2 weight percent based on the total weight of the composition. The concentration range for the agent in the fuel can also be determined by those skilled in the art by determining the partition coefficient of the agent for the fuel or oil and water mixture, and then calculating the amount to add to the fuel or oil to achieve 1 to 5% of the antimicrobial agent in the water that may contaminate the oil or fuel.

The fuel or oil composition may contain typical additives such as detergents, octane boosters, oxygenates, corrosion inhibitors, lubricants, metal deactivators, antioxidants, antiknock agents, dyes, combustion catalysts, burn rate modifiers, deposit control additives, friction modifiers, viscosity modifiers, antiwear additives, pour point depressants, anti-foam agents, seal conditioners, extreme pressure agents, dispersants, and wax crystal modifiers.

According to a third aspect, the invention provides a personal care product comprising about 1 to 5 weight percent of an antimicrobial agent selected from the group consisting of 1,2-cyclohexanedimethanol, 1,4-cyclohexanedimethanol, and 2,2,4,4-tetramethyl-1,3-cyclobutanediol. The agent can also be present in an amount of about 1 to 3 weight percent, based on the total weight of the product.

In one embodiment, 1,2-CHDM, 1,4-CHDM, TMCBD, or a mixture thereof is the only antimicrobial agent in the personal care product.

In one embodiment, the personal care product contains water and the weight percentage of the antimicrobial agent is based on the amount of water in the product.

In another embodiment, the personal care product is anhydrous and the weight percentage of the antimicrobial agent is based on the total weight of the product.

Examples of personal care products according to the invention include hand soaps, hand sanitizers, body washes, shower gels, shampoos, conditioners, face creams, body lotions, underarm deodorants, mouthwash, toothpaste, cosmetics, contact lens solutions, hairstyling products, acne treatment products, fragrances, and foot, sock, or shoe deodorizing compositions.

According to a fourth aspect, the invention provides a medicated product comprising a medicinal substance and about 1 to 5 weight percent of an antimicrobial agent selected from the group consisting of 1,2-cyclohexanedimethanol, 1,4-cyclohexanedimethanol, and 2,2,4,4-tetramethyl-1,3-cyclobutanediol. The agent can also be present in an amount of about 1 to 3 weight percent, based on the total weight of the product.

In one embodiment, 1,2-CHDM, 1,4-CHDM, TMCBD, or a mixture thereof is the only antimicrobial agent in the medicated product.

In one embodiment, the medicated product contains water and the weight percentage of the antimicrobial agent is based on the amount of water in the product.

In another embodiment, the medicated product is anhydrous and the weight percentage of the antimicrobial agent is based on the total weight of the product.

Examples of medicated products according to the invention include acne treatment products, wound care products, and transdermal patches.

Examples of medicinal substances that can be included in the medicated product of the invention include skin rejuvenating products such as salicylic acid, glycolic acid, Vitamin A, Vitamin E, hyaluronic acid, caffeine, aloe vera, Co-enzyme Q10, collagen, and derivatives thereof; anesthetics such as benzocaine or lidocaine; antifungal products such as ketoconazole or fluconozole and the like; anti-inflammatory or anti-itch substances such as hydrocortisone, benadryl and the like, pain medications such as morphine sulfate; and the like, antibiotics, such as amoxicillin, penicillin, trimethoprim, bactrim, sulfamethizole, erythromycin, polymyxin B Sulfate and the like; hormones such as estradiol, progestin, progesterone, testosterone and the like; anti-anxiety medications; anti-depressants or anti-Parikinson's medication, such as selegeline and the like; anti-spasmodic medications such as oxybutynin; anti-convulsive medications such as carbamazepine, anti-motion sickness medication such as scopoloamine; anti-smoking medications such as nicotine; anti-cancer medications such tamoxiphen or 5-fluorouracil, anti-dandruff medications, anti-perspirant medications and actives, and anti-viral medications such as vaccine ingredients.

According to a fifth aspect, the invention provides an animal care product comprising about 1 to 5 weight percent of an antimicrobial agent selected from the group consisting of 1,2-cyclohexanedimethanol, 1,4-cyclohexanedimethanol, and 2,2,4,4-tetramethyl-1,3-cyclobutanediol. The agent can also be present in an amount of about 1 to 3 weight percent, based on the total weight of the product.

In one embodiment, 1,2-CHDM, 1,4-CHDM, TMCBD, or a mixture thereof is the only antimicrobial agent in the animal care product.

In one embodiment, the personal care product contains water and the weight percentage of the antimicrobial agent is based on the amount of water in the product.

In another embodiment, the animal care product is anhydrous and the weight percentage of the antimicrobial agent is based on the total weight of the product.

Examples of animal care products according to the invention include shampoos, conditioners, and fragrances.

According to a sixth aspect, the invention provides a household care product comprising about 1 to 5 weight percent of an antimicrobial agent selected from the group consisting of 1,2-cyclohexanedimethanol, 1,4-cyclohexanedimethanol, and 2,2,4,4-tetramethyl-1,3-cyclobutanediol. The agent can also be present in an amount of about 1 to 3 weight percent, based on the total weight of the product.

In one embodiment, 1,2-CHDM, 1,4-CHDM, TMCBD, or a mixture thereof is the only antimicrobial agent in the household care product.

In one embodiment, the household care product contains water and the weight percentage of the antimicrobial agent is based on the amount of water in the product.

In another embodiment, the household care product is anhydrous and the weight percentage of the antimicrobial agent is based on the total weight of the product.

Examples of household care products according to the invention include surface cleaners, air or surface deodorizers, laundry care products, dishwashing detergents, and rinse aids.

According to a seventh aspect, the invention provides a method for providing residual antimicrobial activity to a surface. The method comprises topically applying the personal care, medicated, animal care, or household care product of the invention to the surface, and optionally removing any excess amounts of the product from the surface.

The treated surface may be the skin or hair of a human or animal, or inanimate objects such as door handles, floors, counter tops, desktops, and furniture.

These steps may be repeated as often as desired, such as 2 to 6 times daily.

In one embodiment, the surface has a biofilm on it before the product is applied.

According to an eighth aspect, the invention provides a method for preventing or reducing odor from the presence of bacteria or fungi on a mammalian surface. The method comprises topically applying the personal care, medicated, or animal care product of the invention to the mammalian surface, and optionally removing any excess amounts of the product from the mammalian surface.

The mammalian surface can be anywhere on the exposed surface of a mammal including hands, feet, underarm, groin, and teeth.

These steps may be repeated as often as desired, such as 2 to 6 times daily.

According to a ninth aspect, the invention provides a method for providing antimicrobial activity to a film, fiber, molded or extruded article, or composite material made of fibers, polymers, adhesives, and/or gypsum. The method comprises incorporating an antimicrobial agent selected from the group consisting of 1,2-cyclohexanedimethanol, 1,4-cyclohexanedimethanol, and 2,2,4,4-tetramethyl-1,3-cyclobutanediol into the film, fiber, molded or extruded article, or composite material during its manufacturing process. The invention could be dissolved in a plasticizer, such as diethylphthalate (DEP) and mixed directly into the powdered plastic material to be extruded or thermoformed during application. Alternatively, the invention could be dissolved in a common solvent or co-solvent along with the polymer, such as cellulose acetate and cast as a thin film to dry. The powder can then be cryogenically ground to form particles of the correct dimensions.

The amount of the antimicrobial agent present in the film, fiber, molded or extruded article, or composite material can vary depending on various factors including the degree of microbial protection desired. Generally, the antimicrobial agent can be present in an amount of about 1 to 5 weight percent, based on the total weight of the composition. The agent can also be present in an amount of about 1 to 3 weight percent, based on the total weight of the composition.

In one embodiment, 1,2-CHDM, 1,4-CHDM, TMCBD, or a mixture thereof is the only antimicrobial agent in the film, fiber, molded or extruded article, or composite material.

In another embodiment, the method of the invention is effective to prevent a biofilm from forming on a surface of the film, fiber, molded or extruded article, or composite material.

This invention can be further illustrated by the following examples of preferred embodiments thereof, although it will be understood that these examples are included merely for purposes of illustration and are not intended to limit the scope of the invention. In the following examples, all percentages are by weight unless otherwise indicated. Additionally, CHDM-D denotes anhydrous 1,4-cyclohexanedimethanol, and CHDM-D90 denotes a mixture of 90 wt % 1,4-CHDM and 10 wt % water.

### EXAMPLES

### Examples 1-7

### Testing for Adequate Preservation of Mixtures

A test for adequate preservation was carried out in accordance with the European Pharmacopea (6.0) and United States Pharmacopea (5.1). The testing involved inoculating a skin cream formulation serving as an emulsion substrate. The skin cream formulation is shown in Table 1.

TABLE 1

| | Wt % |
|---|---|
| Part A: Water Phase | |
| Deionized water | 88.1 |
| Glycerin | 2.0 |
| Carbopol Ultrez 10 Carbomer | 0.2 |
| Part B: Oil Phase | |
| Promulgen D Cetearyl Alcohol (and) Ceteareth-20 | 2.0 |
| Lexemul GDL Glyceryl Dilaurate | 0.5 |
| Cetyl Alcohol | 1.5 |
| Dow Corning 200 Fluid 350 cSt. Dimethicone | 0.2 |
| NutriLayer Oryza Sative (Rice) Bran Oil Extract | 5.0 |
| Part C: Neutralizer | |
| Triethanolamine, 50% in water | 0.5 |

This cream was the emulsion substrate, which formed the base for all further experimentation. Samples were prepared by adding the CHDM, preservative, and/or 1,2-octanediol at the concentration (in wt %) indicated in Table 2. CHDM-D90 is 1,4-CHDM containing 10 wt % of water.

TABLE 2
Emulsion Substrate Additives

| Example | Description |
|---|---|
| 1 | Emulsion substrate (no additives) |
| 2 | Emulsion substrate with 0.75% CHDM-D90 |
| 3 | Emulsion substrate with 1.5% CHDM-D90 |
| 4 | Emulsion substrate with 2.5% CHDM-D90 |
| 5 | Emulsion substrate with 0.3% phenoxyethanol |
| 6 | Emulsion substrate with 0.05% methylparaben |
| 7 | Emulsion substrate with 0.3% 1,2-octanediol |

For Examples 1 through 6, 390.0 g of cream was weighed into a 600-ml beaker. The cream was stirred at room temperature while adding the specified ingredients. Each sample was stirred for 2 hours, then placed in the refrigerator until inoculation.

### Example 1

Water (10.0 g) was added.

### Example 2

CHDM-D90 (3.00 g) and 7.00 g water were added.

### Example 3

CHDM-D90 (6.00 g) and 4.00 g water were added.

### Example 4

CHDM-D90 (10.0 g) was added.

### Example 5

Phenoxyethanol (1.20 g) and 8.80 g water were added.

### Example 6

Methylparaben (0.200 g) and 9.80 water were added.

For Example 7, 179.4 g cream was weighed into a 400-ml beaker. The cream was stirred at room temperature while adding the specified ingredients. Each sample was stirred for 2 hours, then place in the refrigerator until inoculation.

### Example 7

1,2-Octanediol (0.552 g) and 4.05 g water were added.

The samples of Examples 1 through 6 above were challenged with specific organisms (see Table 3) to produce a contamination of between 1.0×10⁵ cfu/g and 1.0×10⁶ cfu/g. The actual inoculation counts resulting from these challenges were immediately determined by diluting in sterile buffered water and (spread plate method) plating for enumeration. The results of these counts for the challenge organisms are shown in Table 3.

TABLE 3

| Challenge Organism | cfu/g |
|---|---|
| A = Pseudomonas aeruginosa ATCC 9027 | 182,000 |
| B = Staphylococcus aureus ATCC 6538 | 184000 |
| C = Candida albicans ATCC 10231 | 202000 |
| D = Escherichia coli ATCC 8739 | 187000 |
| E = Burkholderia cepacia | 179000 |
| F = Aspergillus niger ATCC 16404 | 174000 |

Challenge organisms were prepared in Mueller-Hinton broth, allowed to grow for 72 hours at 35° C.+/−2° C., centrifuged at 2500 rpm for 5 minutes, and the supernatant broth was removed. The microbial pellet was then re-diluted with sterile buffered water to a turbidity that matched previous 1.0×10⁸ cfu/g concentrations of that organism's specific growth curve.

Samples of Example 7 were not challenged with Burkholderia cepacia due to limited test material. Otherwise, they were treated exactly the same as the test samples of Examples 1 through 6.

The test emulsions were maintained within a specific temperature range optimal for the organisms; 35° C.+/2° C. for the bacteria and 22° C.+/−2° C. for the fungi, for the first three days. They were kept at ambient room temperature for the subsequent time periods.

Subculture samples of approximately 1 gram were taken for counts at 7, 14, and 30 days and incubated under optimal conditions and nutrition for no less than 5 days. Subcultures were diluted 1:2, 1:10, 1:100, . . . , 1:10,000 and plated using the spread plate method onto Plate Count Agar; and onto SAB Dextrose Agar in addition for the Candida and Aspergillus species; and incubated as follows: 35° C.+/−2° C. for the Plate Count Agar and 22° C.+/−2° C. for the SAB Dextrose plates of Candida albicans and Aspergillus niger. Negative results were not reported before 7 days incubation and counts were performed after no less than 5 days incubation. Because of the high viscosity of the test emulsion, at least a 1:2 dilution was required to perform the spread plate subcultures. 0-30 counts represent a 1 to 2 dilution, numbers 1-200 a 1:10 dilution; and the rest represent dilutions of 1:100, 1:1000, or 1:10,000. Counts of Candida and Aspergillus species were made on the agar representing the highest count observed, usually the SAB Dextrose.

Counts were adjusted in accordance to the weight of the subculture sample. Results are shown in Table 4.

TABLE 4
Microorganism Counts (cfu/g)

| | Example | | | | | | |
|---|---|---|---|---|---|---|---|
| | 1 | 2 | 3 | 4 | 5 | 6 | 7 |
| Antimicrobial | None | 0.75% | 1.50% | 2.50% | 0.3% PE | 0.05% | 0.3% OD |
| Agent | | CHDM- | CHDM- | CHDM- | | MP | |
| (wt %) | | D90 | D90 | D90 | | | |
| Species | Pseudomonas aeruginosa | | | | | | |
| Days | Counts | | | | | | |
| 0 | 174000 | 174000 | 174000 | 174000 | 174000 | 174000 | 174000 |
| 7 | >100000 | 160 | 140 | 4 | >100000 | 130 | 60 |
| 14 | >100000 | 16 | 24 | 0 | 68000 | 0 | 4 |
| 30 | >100000 | 0 | 0 | 0 | 31000 | 0 | 0 |
| Species | Staphylococcus aureus | | | | | | |
| Days | Counts | | | | | | |
| 0 | 184000 | 184000 | 184000 | 184000 | 184000 | 184000 | 184000 |
| 7 | >100000 | 410 | 0 | 2 | >100000 | 48000 | 80 |
| 14 | >100000 | 30 | 0 | 0 | >100000 | 90 | 40 |
| 30 | >100000 | 6 | 0 | 0 | >100000 | 50 | 0 |
| Species | Candida albicans | | | | | | |
| Days | Counts | | | | | | |
| 0 | 202000 | 202000 | 202000 | 202000 | 202000 | 202000 | 202000 |
| 7 | >100000 | 220 | 110 | 0 | >100000 | 86000 | >100000 |
| 14 | >100000 | 0 | 0 | 0 | >100000 | 7200 | >100000 |
| 30 | >100000 | 0 | 0 | 0 | >100000 | 40 | >100000 |
| Species | Escherichia coli | | | | | | |
| Days | Counts | | | | | | |
| 0 | 187000 | 187000 | 187000 | 187000 | 187000 | 187000 | 187000 |
| 7 | >100000 | 1060 | 0 | 28 | >100000 | 6600 | 1500 |
| 14 | >100000 | 160 | 0 | 0 | >100000 | 510 | 90 |
| 30 | >100000 | 12 | 0 | 0 | >100000 | 30 | 40 |
| Species | Burkholderia cepacia | | | | | | |
| Days | Counts | | | | | | |
| 0 | 179000 | 179000 | 179000 | 179000 | 179000 | 179000 | NT |
| 7 | >100000 | 190 | 4 | 6 | >100000 | 1400 | NT |
| 14 | >100000 | 20 | 0 | 0 | 71000 | 200 | NT |
| 30 | >100000 | 0 | 0 | 0 | 49000 | 26 | NT |
| Species | Aspergillus niger | | | | | | |
| Days | Counts | | | | | | |
| 0 | 174000 | 174000 | 174000 | 174000 | 174000 | 174000 | 174000 |
| 7 | >100000 | 1090 | 1440 | 1270 | >100000 | >100000 | >100000 |
| 14 | >100000 | 150 | 190 | 110 | >100000 | >100000 | >100000 |
| 30 | >100000 | 30 | 18 | 6 | >100000 | >100000 | >100000 |

### Example 8

### Determination of MIC and MLC for 1,4-CHDM and Propylene Glycol

Two antimicrobial endpoints were investigated. The minimum inhibitory concentration (MIC), which identifies the lowest concentration of test material at which growth for a specific organism is inhibited (by a minimum of 3 log units), reflects “biostatic activity”. The minimum lethal concentration (MLC), or lowest concentration that kills the organism and at which subculturing with growth is not possible, reflects “biocidal activity”. 1,4-CHDM and propylene glycol (PG) were added to previously sterilized screw-cap glass tubes and diluted with Peptone Water Broth with 2 wt % Dextrose (=test broth) to the following dilutions (weight percent): 0.05, 0.075, 0.125, 0.25, 0.5, 0.75, 1.25, 2.5, 5.0, 7.5, 10.0, 15.0, 20.0.

Each of 12 sets of the above test tubes were challenged with specific organisms (see Table 5) to produce a contamination of between 1.0×10⁵ cfu/g and 1.0×10⁶ cfu/g in the test broth to which no 1,4-CHDM or propylene glycol had been added. These challenge organisms were prepared in TSB broth, allowed to grow for 72 hours at 35° C.+/−2° C., centrifuged at 2500 rpm for 5 minutes, and the supernatant broth was removed. The microbial pellet was then re-diluted with sterile buffered water to a turbidity that matched previous 1.0×10⁶ cfu/g concentrations of that organism's specific growth curve. This challenge was repeated on day 3. Plate counts of both challenges enumerated to within 1.0×10⁵+/−5,000 cfu/g after 7 days incubation at 35° C.+/−2° C.

These tubes were mixed well by inversion (minimum of 25 inversions) and incubated at 35° C.+/−2° C. The tubes were inverted again (minimum of 25 times) on the third and seventh day of incubation and prior to each subculture.

These tubes were observed for turbidity, in some cases photographed, and subculture samples of approximately 0.1 ml were taken for counts at 7, 14, 30, and 60 days. These subcultures were incubated under optimal conditions and nutrition for no less than 5 days. Subcultures of 0.1 ml, undiluted and diluted 1:10, 1:100, . . . , and 1:10,000, were transferred to plates using the spread plate method onto Plate Count Agar; and SAB Dextrose Agar in addition for the Candida and Aspergillus species; and incubated as follows: 35° C.+/−2° C. for the Plate Count Agar and 22° C.+/−2° C. for the SAB Dextrose plates of Candida albicans and Aspergillus niger. Negative results were not reported before 7 days incubation and counts were performed after no less than 5 days incubation. Counts of Candida and Aspergillus species were made on the agar representing the highest count observed, usually the SAB Dextrose. MIC levels were reported for the greatest dilution that showed a minimum of 3 log decrease in cfu/g and MLC levels were reported for the greatest dilution that demonstrated no growth undiluted. Results of the 30-day subcultures are shown in Table 5.

TABLE 5

| | MIC CHDM | MLC CHDM | MIC PG | MLC PG |
|---|---|---|---|---|
| SPECIES | 30 DAYS | 30 DAYS | 30 days | 30 days |
| Staphylococcus | 5 wt % | >20 wt % | 20 wt % | >20 wt % |
| aureus | | | | |
| ATCC 25923 | | | | |
| Streptococcus | 2.5 | 20 | 20 | >20 |
| pyogenes | | | | |
| Streptococcus | 2.5 | >20 | 20 | >20 |
| faecalis | | | | |
| Escherichia coli | 2.5 | >20 | 10 | >20 |
| ATCC 25922 | | | | |
| Proteus vulgaris | 2.5 | >20 | 20 | >20 |
| Pseudomonas | 2.5 | 20 | 10 | >20 |
| aeruginosa | | | | |
| ATCC 27853 | | | | |
| Burkholderia | 1.25 | 20 | 10 | >20 |
| cepacia | | | | |
| ATCC 25416 | | | | |
| Bacillus subtilis | 2.5 | >20 | 10 | >20 |
| ATCC 6633 | | | | |
| Aspergillus niger | 2.5 | >20 | 20 | >20 |
| ATCC 16404 | | | | |
| Candida albicans | 2.5 | 20 | 20 | >20 |
| ATCC 10231 | | | | |
| Penicillium | 5 | >20 | >20 | >20 |
| notatum | | | | |
| ATCC 36740 | | | | |

### Example 9

### Water Extraction of 1,4-CHDM from Biodiesel

A solution of 59.5 wt % 1,4-CHDM in ethoxydiglycol (Eastman DE solvent) was prepared by weighing into a vial 1.36 g ethoxydiglycol and 2.00 g 1,4-CHDM. The mixture was heated to 55° C. to melt the 1,4-CHDM, mixed on a vortex mixer, then placed on a rocker mixer overnight at room temperature to allow all of the 1,4-CHDM to dissolve.

1,4-CHDM was added to soy biodiesel as follows: 60.00 g soy biodiesel was weighed into 4-oz jar. 0.0246 g of the above CHDM/DE solution was weighed into the biodiesel. The sample was mixed using a magnetic stirrer. There was no visible separation after mixing and the biodiesel remained clear. The theoretical amount of CHDM in the biodiesel is 0.024 wt %. 5.00 g was removed from the sample for analysis.

To make a biodiesel mixture containing 0.90% water, 0.50 g deionized water was added to the remaining 55.0 g 1,4-CHDM/DE/biodiesel mixture and mixed with a magnetic stirrer. The mixture became cloudy when stirred and also contained visible water droplets. The mixture was allowed to sit undisturbed for 3 days, during which time a white aqueous layer appeared on the bottom and the biodiesel (top layer) still remained cloudy. The mixture was centrifuged to separate as much water as possible from the biodiesel. After centrifuging, the biodiesel was again clear with an aqueous layer on the bottom. The 1,4-CHDM/DE/biodiesel mixtures before and after water extraction and the bottom aqueous layer were analyzed by gas chromatography for 1,4-CHDM. The 1,4-CHDM in the 1,4-CHDM/DE/biodiesel mixture before and after extraction was found to be at the lower detection limit of about 0.015 to 0.02%. The aqueous bottom layer was found to contain 0.38 wt % 1,4-CHDM. This indicates that the water extracted a portion of the 1,4-CHDM that was added to the biodiesel. Thus, 1,4-CHDM that is present in the water would inhibit microbial growth in the water or at the biodiesel/water interface.

### Example 10

### 1,4-CHDM Challenge Testing Against Pathogenic Fungi

Both Microsporum canis and Trichophyton rubrum were grown on Sabouraud dextrose broth, while Malassezia fufur was grown in Sabouraud dextrose broth supplemented with 2% (v/v) of olive oil and 0.2% (v/v) of Tween™ 80; incubation was at 22°±2° C. under continuous agitation by stirring for 10 days. The organisms were grown to a density of between 1.0×10³ to 1.0×10⁴ cfu/g. The actual inoculation cell density of these challenges were determined by diluting in sterile buffer water and (spread-plate method) plating for enumeration. The results of these counts for the challenge organisms are shown in Table 6.

TABLE 6
Challenge Organisms and Inoculation Cell Density

| | |
|---|---|
| Microsporum canis (ATTC 9084) | 46,000 cfu/g |
| Trichophyton rubrum (ATCC 10218) | 1,300 cfu/g |
| Malassezia furfur (ATCC 96809) | ND* |

*Note:
M. furfur culture was very turbid and viable but plating onto Sabouraud dextrose agar (supplemented with olive oil-Tween ™ 80) for enumeration did not give countable colonies.

Challenge organisms were used to inoculate tubes of 1,4-CHDM-D90 test concentrations prepared in Sabouraud dextrose broth (or Sabouraud dextrose broth supplemented with olive oil-Tween™ 80 with M. furfur). The inoculations were in the amount of 1.5 mL aliquots of each culture with static incubation at 22±2° C. Subcultures were made at 3-, 14- and 30-day increments. All challenges were conducted in triplicate. In the case of M. canis, the growth response was assessed by the visual presence/absence of growth in the tubes; in the case of T. rubrum, a respiratory (redox) dye (0.2% w/v aqueous INT solution: 2-[4-iodophenyl)-3-4-nitrophenyl]-5-phenyl tetrazolium chloride) was added to the tubes, turning red if the organism was viable; and, finally, in the case of M. furfur, the growth response was assessed based upon pellicle formation in the tubes at the meniscus. The test formulations and challenge test results, as well as 30-day MICs (Minimum Inhibitory Concentration), are in Tables 7-9 for the three organisms using the following growth rating system:

### Growth Rating

0: No visible growth
1: Some growth in tube
2: Moderate growth in tube
3: Good growth in tube
4: Extreme growth in tube

TABLE 7
1,4-CHDM-D90 Challenge Data for Microsporum canis
for 3-, 14-, and 30-day Exposures

| | Growth | |
|---|---|---|
| | Rating | 30-Day |
| Test Concentration | 3/14/30 Days | MIC |
| 1,4-CHDM-D90: 5% | 0/0/0 | |
| 1,4-CHDM-D90: 2.5% | 2/0/0 | X |
| 1,4-CHDM-D90: 1.25% | 2/1/1 | |
| 1,4-CHDM-D90: 0.5% | 2/1/1 | |
| 1,4-CHDM-D90: 0% | 2/1/1 | |

TABLE 8
1,4-CHDM-D90 Challenge Data for Trichophyton rubrum
for 3-, 14-, and 30-day Exposures

| | Growth | |
|---|---|---|
| | Rating | 30-Day |
| Test Concentration | 3/14/30 Days | MIC |
| 1,4-CHDM-D90: 5% | 1/0/0 | |
| 1,4-CHDM-D90: 2.5% | 0/0/0 | |
| 1,4-CHDM-D90: 1.25% | 0/0/0 | X |
| 1,4-CHDM-D90: 0.5% | 1/1/1 | |
| 1,4-CHDM-D90: 0% | 1/0/3 | |

TABLE 9
1,4-CHDM-D90 Challenge Data for Malassezia furfur
for 3-, 14-, and 30-day Exposures

| | Growth | |
|---|---|---|
| | Rating | 30-Day |
| Test Concentration | 3/14/30 Days | MIC |
| 1,4-CHDM-D90: 5% | 0/0/0 | |
| 1,4-CHDM-D90: 2.5% | 0/0/0 | |
| 1,4-CHDM-D90: 1.25% | 0/0/0 | X |
| 1,4-CHDM-D90: 0.5% | 0/1/2 | |
| 1,4-CHDM-D90: 0% | 0/3/3 | |

### Example 11

### Formulation 1, Table 10, Mascara with 1,4-CHDM

A mascara formulation was prepared by mixing and stirring together by hand the ingredients of Part I (listed in Table 10) in a 150-mL beaker at 60° C. until the ingredients dissolved.

Part II (listed in Table 10) was melted together in a 100-mL beaker with a magnetic stirrer/hotplate at 75° C. It was allowed to cool to 60° C.

While both Parts I and II were at 60° C., they were blended together by hand. Finally, CHDM-D90 was added to the Part I and II mixture by hand.

TABLE 10
(Formulation 1, Example 11)

| | Amount | Weight Percent of Total |
|---|---|---|
| | (g) | (Part I + Part II + CHDM) |
| Part I Ingredients | | |
| Deionized Water | 34.20 | 67.19 |
| Xanthum Gum | 0.50 | 0.98 |
| Panthenol | 0.25 | 0.49 |
| Glycerol | 1.00 | 1.97 |
| Gum Arabic | 1.00 | 1.97 |
| Part II Ingredients | | |
| Stearic Acid | 2.50 | 4.91 |
| Candelilla Wax | 0.75 | 1.47 |
| Steareth-20 | 0.43 | 0.84 |
| Cetyl alcohol (1-hexadecanol) | 0.42 | 0.83 |
| Beeswax | 2.25 | 4.42 |
| Carnuba Wax | 1.35 | 2.65 |
| Unipure Black LC989 | 5.00 | 9.82 |
| Dimethicone | 0.25 | 0.49 |
| SUBSTOTAL | 49.90 | 98.03 |
| CHDM-D90 | 1.00 | 1.97 |
| TOTAL | 50.90 | 100.00 |

### Example 12

### Formulation 2, Progestin Patch with 1,4-CHDM

An adhesive patch containing progestin was prepared by dissolving 30.00 g of Eastman AQ® 2350 polymer in 170.00 g of water at 55° C. using a rotary stirring device to form a first mixture. 1.00 g of progestin was dissolved in 9.00 g of CHDM-D90 in a vial using a shaking mixer to form a second mixture. 10.03 g of the first mixture was mixed with 0.11 g of the second mixture to form a third mixture. 2.16 g of the third mixture was combined with 0.23 g of ethanol to form a fourth mixture. The fourth mixture was cast onto a polyethylene sheet to form an adhesive layer containing progestin.

The adhesive formulation on a dry basis contained 93.79 wt % of AQ 2350, 0.68 wt % of progestin, and 5.53 wt % of 1,4-CHDM. The formulation had a specific gravity of 0.9 g/cc.

Each patch was 10 cm×10 cm with a 2-micron thick adhesive layer. Each patch contained on a dry basis 1.23 mg of progestin.

### Example 13

### Formulation 3, Table 11, Antimicrobial Cellulosic Fibers with 1,4-CHDM

Antimicrobial cellulosic fibers containing 1,4-CHDM were prepared by mixing the ingredients of Table 11 in the proportions shown. The mixture was added to a sealed glass jar and placed on a roller mixer until completely dissolved. The polymer solution (dope) was then cast on glass and allowed to air dry followed by drying in a vacuum oven at 50° C. overnight. Chips were made of the dried clear thin films and fed into a fiber spinning device at 180-260° C. to form the fibers.

TABLE 11
(Formulation 3, Example 13)

| | Amount | |
|---|---|---|
| Ingredient | (g) | Weight Percent |
| Pellets of Eastman CAP-141-20 plasticized | 120.08 | 20.88 |
| with Resoflex R296 plasticizer (10%) | | |
| CHDM-D | 5.00 | 0.87 |
| Methyl acetate | 140.0 | 24.34 |
| Ethyl acetate | 220.0 | 38.26 |
| Ethanol, 3A | 90.0 | 15.65 |
| TOTAL | 575.08 | 100.00 |

The fibers were clear, colorless, strong, and had low static charge. The fibers had the composition listed in Table 12.

TABLE 12
(Formulation 4, Example 13)

| | Amount | |
|---|---|---|
| Ingredient | (g) | Weight Percent |
| CAP-141-20 | 108.07 | 86.40 |
| Resoflex R296 | 12.01 | 9.60 |
| CHDM-D | 5.00 | 4.00 |
| TOTAL | 125.08 | 100.00 |

### Example 14

### Formulation 5, Molded Plastic Part with 1,4-CHDM

A molded plastic part was prepared by mixing 16.25 g of diethyl phthalate (DEP) with 5 g of CHDM-D (anhydrous 1,4-CHDM) until the DEP dissolved. 85 g of CAP-141-20 powder was added to the mixture in a sealed glass jar and allowed to mix on a roller mixer overnight. The mixture was molded into a plastic sheet in a heated press set at 240° C. for 60 seconds and 420 pounds per square inch.

### Example 15

### Formulations 6-9, Table 13, Antimicrobial Surface Spray

C₁₂₋₁₆ alkyl polyglycoside surfactant (Plantaren® 1300) was mixed with water, ethanol, CHDM-D, and ethylene glycol monobutyl ether (Eastman™ EB) in a jar and sealed. The proportions of the ingredients are shown in Table 13. The mixture was then placed on a roller mixer to dissolve overnight. Four samples were prepared. The pH was adjusted to 8.0 in two of the samples using triethanolamine at the amount shown in Table 13.

TABLE 13
(Formulations 6-9, Example 15)

| | Sample 1 | Sample 2 | Sample 3 | Sample 4 |
|---|---|---|---|---|
| Ingredient | (%) | (%) | (%) | (%) |
| Plantaren ® 1300 | 2.5 | 2.5 | 2.5 | 2.5 |
| Ethanol | 1.0 | 1.0 | 1.0 | 1.0 |
| Eastman ™ EB | 8.0 | 8.0 | 8.0 | 8.0 |
| CHDM-D | 1.0 | 1.0 | 2.5 | 2.5 |
| Triethanolamine | 0 | 0.1 | 0 | 0.1 |
| Water | 87.5 | 87.4 | 86.0 | 85.9 |
| TOTAL | 100 | 100 | 100 | 100 |
| pH | 5.5 | 8.0 | 5.5 | 8.0 |

Pure cultures of the four (4) test microorganisms (i.e., Bacillus subtilis ATCC 6633: gram-positive, spore-forming bacterium; Candida albicans ATCC 10231: fungus—yeast; Burkholderia cepacia ATCC 25416: gram-negative bacterium; and Staphylococcus aureus ATCC 25923: gram-positive bacterium) were grown in liquid medium (Trypticase Soy Broth for the bacteria @ 35° C. and Sabouraud Dextrose Broth for the fungus @ 22° C.). Both the B. cepacia and S. aureus are pathogenic microorganisms (i.e., BSL-2, Biosafety Level 2); all manipulations were performed in a Class II biosafety cabinet and all materials were decontaminated by use of steam-sterilization. After growth for 48-72 hours, standard plate counts were performed to determine cell density; the cell density of the individual microbial cultures were as follows: B. subtilis was 3.4×10¹⁰ cfu/mL, C. albicans was 1.3×10⁷ cfu/mL, B. cepacia was 8.0×10¹⁰ cfu/mL, and S. aureus was 10⁶ cfu/mL, respectively. The cultures were diluted by 1:10⁵ or 1:10⁶ in sterile buffer water in order to achieve a survivor cell count (cfu or colony-forming unit) that could be determined by contact (RODAC) plates; 10-μL aliquots of each diluted culture were carefully transferred via serological pipette to pre-sterilized, glass slides (50 mm×55 mm) contained with sterile, plastic Petri dishes (135-mm diameter). The aliquots were spread evenly out over the slide surface via sterile, plastic inoculating loops, each dish was then given two (2) sprays (˜0.28 g) of an individual formulation in Table 13; at pre-selected exposure times (i.e., 30 minutes, 90 minutes, 8 hours, 24 hours, 1 week) each duplicate slide for the formulation was carefully pressed with a Standard Methods Agar contact plate (RODAC plates) and incubated at 35° C. (for bacteria) or 22° C. (for fungi) for a period of 5-7 days, then counted visually. During the exposure periods, the slide-Petri dish assemblies were held in humidified, sealed incubators at room temperature. A control was run for each of the test organisms involving 10-μL aliquots of the same diluted cultures.

All of the tests were negative (0 cfu) for all formulations with each organism over the entire range of exposure times; the controls, other than for that of S. aureus, were robust (i.e., ˜300 cfu). (S. aureus may be more susceptible to desiccative injury than the other test organisms.) Overall, these results show that the test organisms did not survive the exposure to the formulations, suggesting that the formulations are all potent surface disinfectants, regardless of the exposure time regimen (i.e., immediate kill).

### Example 16

### Formulation 10, Table 14, Wound Care Spray

A wound care spray was prepared by dissolving triclosan (5-chloro-2-(2,4-dichlorophenoxy)phenol) in ethanol and water to form a first mixture. Eastman AQ® 48 polymer was dissolved in water by stirring at 80° C. After dissolution, triethyl citrate and glycerin were added to the AQ 48 solution to form a second mixture. The first mixture was then added to the second mixture with stirring to form a wound care spray composition. The amounts of the ingredients are shown in Table 14.

TABLE 14
(Formulation 10, Example 16)

| | | Weight |
|---|---|---|
| | Amount | Percentage |
| Ingredient | (g) | (%) |
| First Mixture | | |
| Ethanol | 37.72 | 29.64 |
| Triclosan | 0.05 | 0.04 |
| CHDM-D90 | 5.00 | 3.93 |
| Second Mixture | | |
| Water | 54.38 | 42.73 |
| Glycerol | 1.43 | 1.12 |
| AQ 48 (Polyester-5) | 27.26 | 21.42 |
| Triethyl citrate | 1.43 | 1.12 |
| TOTAL | 127.27 | 100.00 |

The composition had a Brookfield Viscosity of 21 cP. The composition can be sprayed on a skin surface using a pump sprayer or pressurized sprayer.

### Example 17

### Formulation 11, Acne Skin Care Spray

Topical formulations to treat acne are often irritating to the skin. The two over the counter medications used to treat acne: salicylic acid and benzoyl peroxide can leave the skin irritated and red. Formulation 11 could be used as a protective spray in between harsher topical acne treatments. The formulation has a near neutral pH (pH 6.0), a polymer to protect the skin from further irritation, and CHDM-D90 to provide an ingredient that has been shown to be effective against one of the bacteria that causes acne lesions, staphalacoccus aureus.

### Acne Skin Care Spray Formulation 11

### Step I

Preparation of AQ 48 (polyester-5) 32% dispersion

| | Amount | |
|---|---|---|
| | (g) | Percent |
| AQ 48 (Polyester-5) | 128.00 | 32.00 |
| Deionized water | 272.00 | 68.00 |
| Subtotal | 400.00 | 100.00 |

The above components were added to a glass jar and sealed. They were placed on a roller mixer until dissolved.

### Step II

Add the following ingredients in the order listed.

| | Amount | |
|---|---|---|
| | (g) | Percent |
| 32% dispersion of AQ 48 (from Step I) | 60.65 | 44.92 |
| Triethyl citrate | 1.02 | 0.76 |
| Ethanol | 17.88 | 13.24 |
| CHDM-D90 | 5.00 | 3.70 |
| Silsoft 870 (PEG-12 dimethicone) | 0.33 | 0.24 |
| Deionized Water | 50.14 | 37.14 |
| Total | 135.02 | 100.00 |

### Example 18

### Formulation 12, Table 15, Foot/Shoe/Sock Odor Reducing Spray

A foot/shoe/sock odor reducing spray was prepared by mixing the ingredients in Table 15 directly in a spray bottle. The bottle was then sealed and physically shaken until all of the ingredients were in solution.

TABLE 15
(Formulation 12, Example 18)

| | | Weight |
|---|---|---|
| | Amount | Percentage |
| Ingredient | (g) | (%) |
| Isopropyl Alcohol | 69 | 63.23 |
| Deionized Water | 29 | 26.57 |
| CAP-504-02 | 1 | 0.92 |
| Ethanol | 5 | 4.58 |
| CHDM-D | 3 | 2.75 |
| Benzalkonium Chloride | 0.13 | 0.12 |
| Fragrance (Fresh Ginger Lime BBW Type) | 2 | 1.83 |
| TOTAL | 109.13 | 100 |

### Example 19

### Formulation 13, Table 16, Fragrant Foot Deodorizer/Deodorant

A foot deodorizer/deodorant was prepared by mixing the ingredients in Table 16 directly in a plastic bottle. The bottle was shaken at 35° C. using a shaker incubator until all of the ingredients dissolved.

TABLE 16
(Formulation 13, Example 19)

| | | Weight |
|---|---|---|
| | Amount | Percentage |
| Ingredient | (g) | (%) |
| White Tea and Ginger ™ Body Splash | 25.00 | 93.46 |
| CHDM-D | 1.25 | 4.67 |
| CAP-504-02 | 0.50 | 1.87 |
| TOTAL | 26.75 | 100.00 |

### Example 20 Formulation 14, Table 17, Deodorizer with CHDM and Fragrance

A skin or fabric deodorizer was prepared by mixing the ingredients in Table 17 directly in a plastic bottle. The bottle was shaken until all of the ingredients dissolved.

TABLE 17
(Formulation 14, Example 20)

| | | Weight |
|---|---|---|
| | Amount | Percentage |
| Ingredient | (g) | (%) |
| Fragrance (Rain Drops Type) | 4.20 | 4.19 |
| Ethanol | 75.00 | 74.85 |
| Water | 16.00 | 15.97 |
| CHDM-D90 | 5.00 | 4.99 |
| TOTAL | 100.20 | 100.00 |

### Example 21

### Table 18, Formulation 15, Deodorizer with CHDM and Fragrance

A skin or fabric deodorizer was prepared by mixing the ingredients in Table 18 directly in a plastic bottle. The bottle was shaken at 35° C. using a shaker incubator until all of the ingredients dissolved.

TABLE 18
(Formulation 15, Example 21)

| | | Weight |
|---|---|---|
| | Amount | Percentage |
| Ingredient | (g) | (%) |
| Fragrance (Rain Drops Type) | 4.00 | 9.61 |
| Ethanol | 31.00 | 74.47 |
| Water | 5.00 | 12.01 |
| CAP-504-02 | 0.81 | 1.95 |
| CHDM-D90 | 0.82 | 1.97 |
| TOTAL | 41.63 | 100.00 |

### Example 22

### Antimicrobial Fragrance Deodorizer

A skin or fabric deodorizer was prepared by mixing the ingredients in Table 19 directly in a plastic bottle. The bottle was shaken until all of the ingredients dissolved.

TABLE 19
(Formulation 16, Example 22)

| | | Weight |
|---|---|---|
| | Amount | Percentage |
| Ingredient | (g) | (%) |
| Fresh Cucumber ™ Body Splash | 25.00 | 96.15 |
| CHDM-D90 | 1.00 | 3.85 |
| TOTAL | 26.00 | 100.00 |

To test the commercial fragrance as a deodorizer and to compare it to the formulation in Table 19, Example 22, Fresh Cucumber™ Body Splash was weighed out into aluminum weighing dish containing a section of cotton cloth which had been used for cleaning the floor and the mixture allowed to evaporate at room temperature for 24 hours. An identical amount of the formulation in Table 19 was also weighed into an aluminum weighing dish containing a section of cotton cloth that had been used for cleaning the floor and the mixture allowed to evaporate at room temperature. After 24 hours, the fragrance of the two samples left in the aluminum dishes were compared. A cucumber fragrance was noticeable in the formulation from Table 19 and no malodor was present. Without CHDM-D90, the Fresh Cucumber™ Body Splash did not retain a cucumber fragrance and a musty smell was prevalent.

### Example 23

### Formulation 17, Table 20, Unscented Antiperspirant Fast Drying Spray with 1,4-CHDM

An antiperspirant spray was prepared by mixing the ingredients in Table 20 directly in a plastic bottle. The bottle was shaken at 35° C. using a shaker incubator until all of the ingredients dissolved.

TABLE 20
(Formulation 17, Example 23)

| | | Weight |
|---|---|---|
| | Amount | Percentage |
| Ingredient | (g) | (%) |
| Ethanol | 8.00 | 62.60 |
| Water | 22.00 | 17.66 |
| Triacetin | 1.00 | 0.80 |
| Aluminum Chlorohydrate | 20.00 | 16.05 |
| CAP-504-02 | 1.60 | 1.28 |
| CHDM-D90 | 1.00 | 0.80 |
| Propylene Carbonate | 1.00 | 0.80 |
| TOTAL | 124.60 | 100.00 |

### Example 24

### Formulation 18, Table 21, Scented Antiperspirant Spray with 1,4-CHDM

The formulation in Table 21 was placed in a pump spray bottle and sprayed as a fine mist to the underarms.

TABLE 21
(Formulation 18, Example 24)

| | | Weight |
|---|---|---|
| | Amount | Percentage |
| Ingredient | (g) | (%) |
| Mixture I: | | |
| Water | 100.00 | 81.97 |
| Aluminum Chlorohydrate | 20.00 | 16.39 |
| CHDM-D90 | 1.00 | 0.82 |
| Propylene Carbonate | 1.00 | 0.82 |
| Subtotal | 122.00 | 100.00 |
| Formulation 18: | | |
| Mixture I (above) | 11.61 | 89.51 |
| Fragrance Oil, Jasmine Tuberose | 1.12 | 8.63 |
| Steareth-20 | 0.12 | 0.93 |
| Steareth-2 | 0.12 | 0.93 |
| Total | 12.97 | 100.00 |

### Example 25

### Formulation 19, Table 22, Unscented Antiperspirant Spray with 1,4-CHDM

The formulation in Table 22 was placed in a pump spray bottle and sprayed as a fine mist to the underarms.

TABLE 22
(Formulation 19, Example 25)

| | | Weight |
|---|---|---|
| | Amount | Percentage |
| Ingredient | (g) | (%) |
| Water | 100.00 | 81.97 |
| Aluminum Chlorohydrate | 20.00 | 16.39 |
| CHDM-D90 | 1.00 | 0.82 |
| Propylene Carbonate | 1.00 | 0.82 |
| TOTAL | 122.00 | 100.00 |

### Example 26

### Formulation 20, Table 23, Roll-On with 1,4-CHDM

An underarm roll-on composition was prepared by heating the ingredients of Mixture I at 70° C. until they dissolved. Separately, the ingredients of Mixture II were heated at 70° C. until they dissolved. Mixture II was added to Mixture I. While the Mixtures I and II were still hot, aluminum chlorohydrate was added with mixing. The blend was then sheared in a high shear blender. Mixture IV was added to the blend while the blend was cooled. The ingredients and amounts are shown in Table 23.

TABLE 23
(Formulation 20, Example 26)

| | | Weight |
|---|---|---|
| | Amount | Percentage |
| Ingredient | (g) | (%) |
| Mixture I | | |
| Stereath-20 | 1.00 | 0.78 |
| Deionized Water | 40.00 | 31.25 |
| CHDM-D90 | 3.00 | 2.34 |
| Mixture II | | |
| Stereath-2 | 1.00 | 0.78 |
| Sweet Almond Oil | 10.00 | 7.81 |
| Mixture III | | |
| Aluminum Chlorohydrate | 30.00 | 23.44 |
| Mixture IV | | |
| DI Water | 40.00 | 31.25 |
| Fragrance | 3.00 | 2.34 |
| TOTAL | 128.00 | 100.00 |

The composition had a Brookfield Viscosity of 421 cP with spindle #3 at 22° C.

### Example 27 (Comparative)

### Formulation 21, Table 24, Roll-On without 1,4-CHDM

An underarm roll-on composition was prepared by heating the ingredients of Mixture I at 70° C. until they dissolved. Separately, the ingredients of Mixture II were heated at 70° C. until they dissolved. Mixture II was added to Mixture I. While the Mixtures I and II were still hot, aluminum chlorohydrate was added with mixing. The blend was then sheared in a high-shear blender. Mixture IV was added to the blend while the blend was cooled. The ingredients and amounts are shown in Table 24.

TABLE 24
(Formulation 21, Example 27 (Comparative))

| | | Weight |
|---|---|---|
| | Amount | Percentage |
| Ingredient | (g) | (%) |
| Mixture I | | |
| Stereath-20 | 1.00 | 0.78 |
| Deionized Water | 40.00 | 31.25 |
| Propylene Glycol | 3.00 | 2.34 |
| Mixture II | | |
| Stereath-2 | 1.00 | 0.78 |
| Sweet Almond Oil | 10.00 | 7.81 |
| Mixture III | | |
| Aluminum Chlorohydrate | 30.00 | 23.44 |
| Mixture IV | | |
| DI Water | 40.00 | 31.25 |
| Fragrance | 3.00 | 2.34 |
| TOTAL | 128.00 | 100.00 |

The composition had a Brookfield Viscosity of 636 cP with spindle #3 at 22° C.

Samples from Formulation 20 and Formulation 21 were allowed to sit at room temperature for 10 months. The sample from Example 26 (with 1,4-CHDM) did not phase separate, while the sample from Example 27 (with PG) did.

### Example 28

### Formulation 22, Table 25, Anhydrous Antiperspirant Stick with 1,4-CHDM

An anhydrous antiperspirant stick was prepared by heating the ingredients in Table 25, Formulation 22 to 75° C. in a double boiler with stirring. The sample was cooled to 65° C. at which time, fragrance was added with stirring. Samples were poured into deodorant containers to the top and immediately covered to prevent evaporation of volatile components.

TABLE 25
(Formulation 22, Example 28)

| | | Weight |
|---|---|---|
| | Amount | Percentage |
| Ingredient | (g) | (%) |
| Stearyl Alcohol (1-octadecanol) | 10.00 | 16.39 |
| Decamethylcyclopentasiloxane | 10.00 | 16.39 |
| CHDM-D90 | 1.00 | 1.64 |
| PPG-14 butyl ether | 26.50 | 43.44 |
| Aluminum Chlorohydrate | 10.00 | 16.39 |
| Talc | 2.50 | 4.10 |
| Fragrance | 1.00 | 1.64 |
| TOTAL | 61.00 | 100.00 |

### Example 29

### Table 26, Formulation 23, Non-Whitening Antiperspirant Stick with 1,4-CHDM

An antiperspirant stick was prepared by melting the ingredients of Part I (listed in Table 26, Formulation 23) together in a double boiler at 75° C. with stirring. The mixture was cooled to 65° C. The ingredients of Part II were mixed and allowed to soak together for 15 minutes. They were then heated to 65° C. Part II was stirred into Part I while both were at 65° C. The mixture was cooled to 60° C. while stirring, and then poured into deodorant containers and covered immediately to cool. The antiperspirant was non-whitening when applied to the underarms and prevented malodor from developing.

TABLE 26
(Formulation 23, Example 29)

| | | Weight |
|---|---|---|
| | Amount | Percentage |
| Ingredient | (g) | (%) |
| Part I | | |
| Lumulse GMS-A | 1.94 | 1.98 |
| Stearyl Alcohol | 18.43 | 18.86 |
| Castor Was MP-70 | 1.94 | 1.98 |
| Talc, USP | 2.5 | 2.56 |
| CHDM-D | 2.05 | 2.10 |
| Part II | | |
| PPG-14 butyl ether | 3.88 | 3.97 |
| Aluminum Zirconium | 19.5 | 19.95 |
| Tetrachlorohydrex Gly | | |
| AAZG-7167 | | |
| Fragrance (Cool Citrus Basil, BBW Type) | 1.94 | 1.98 |
| Dow Corning 245 Fluid | 45.56 | 46.61 |
| TOTAL | 97.74 | 100.00 |

### Example 30

### Table 27, Formulation 24, Clear Scented Deodorant Stick with 1,4-CHDM

A deodorant stick was prepared by first mixing the ingredients of Part I (listed in Table 27, Formulation 24) at 50° C. until they dissolved to form a Part I solution.

Separately, Part IIA liquids (listed in Table 27) were added together and heated to 97° C. Dibenzilidene sorbital (solid) was then added, and the mixture was stirred until it dissolved to form a Part IIA solution and was kept hot at 85° C.

Meanwhile, Steareth-2 and Steareth-20 were melted at 85° C. in the presence of C₁₂-C₁₅ alkyl benzoate and Silsoft 305 to form a Part IIB solution.

The Part IIA solution was then added to the Part IIB solution, followed by the ingredients of Part IIC of fragrance, AMP-95, and Dow Corning 245 Fluid. The mixture was cooled to 60° C. and poured into deodorant containers to form a clear colorless deodorant stick.

TABLE 27
(Formulation 24, Example 30)

| | | Weight |
|---|---|---|
| | Amount | Percentage |
| Ingredient | (g) | (%) |
| Part I Solution | | |
| Aluminum Chlorohydrate | 30.00 | 30.00 |
| Propylene Glycol | 70.00 | 70.00 |
| TOTAL | 100.00 | 100.00 |
| Part IIA | | |
| Part I Solution | 54.05 | 55.80 |
| CHDM-D (liquid) | 3.38 | 3.49 |
| Propylene Carbonate (liquid) | 2.03 | 2.10 |
| Dipropylene Glycol (liquid) | 2.03 | 2.10 |
| Dibenzilidine Sorbital (solid) | 1.50 | 1.55 |
| Part IIB | | |
| Steareth-20 | 10.14 | 10.47 |
| Steareth-2 | 4.73 | 4.88 |
| C₁₂-C₁₅ Alkyl Benzoate | 2.03 | 2.10 |
| Silsoft 305 | 2.70 | 2.79 |
| Part IIC | | |
| Fragrance Oil | 6.76 | 6.98 |
| AMP-95 | 0.75 | 0.77 |
| Dow Corning 245 Fluid | 6.76 | 6.98 |
| TOTAL | 96.86 | 100.00 |

### Example 31

### Table 28, Formulation 25, Unscented Clear Deodorant Stick with 1,4-CHDM

A deodorant stick was prepared by first adding the liquid ingredients of Part I (listed in Table 28, Formulation 25) together and heated to 95° C. Dibenzilidene sorbital was then added until it dissolved. The solution was kept at 85° C.

Meanwhile, Steareth-2 and Steareth-20 of Part II were melted at 85° C. in the presence of C₁₂-C₁₅ alkyl benzoate, Silsoft 305, and propylene glycol to form a Part II solution.

The Part II solution was then added to the Part I solution and cooled while stirring with a magnetic stir bar to 82° C. and poured into deodorant containers.

TABLE 28
Example 31, Formulation 25

| | | Weight |
|---|---|---|
| | Amount | Percentage |
| Ingredient | (g) | (%) |
| Part I | | |
| CHDM-D (liquid) | 4.00 | 4.46 |
| Propylene Carbonate (liquid) | 8.00 | 8.92 |
| Dipropylene Glycol (liquid) | 8.00 | 8.92 |
| Dibenzilidine Sorbital (solid) | 3.70 | 4.12 |
| Subtotal | 23.70 | 26.42 |
| Part II | | |
| Steareth-20 | 12.00 | 13.38 |
| Steareth-2 | 6.00 | 6.69 |
| C₁₂-C₁₅ Alkyl Benzoate | 6.00 | 6.69 |
| Silsoft 305 | 2.00 | 2.23 |
| Propylene Glycol | 40.00 | 44.59 |
| TOTAL | 89.70 | 100.00 |

### Example 32 (Comparative)

### Table 29, Formulation 26, Unscented Clear Deodorant Stick without 1,4-CHDM

A deodorant stick was prepared by first adding the liquid ingredients of Part I (listed in Table 29, Formulation 26) together and heated to 95° C. Dibenzilidene sorbital was then added until it dissolved. The solution was kept at 85° C.

Meanwhile, Steareth-2 and Steareth-20 of Part II were melted at 85° C. in the presence of C₁₂-C₁₅ alkyl benzoate, Silsoft 305, and propylene glycol to form a Part II solution.

The Part II solution was then added to the Part I solution and cooled while stirring with a magnetic stir bar to 82° C. and poured into deodorant containers.

TABLE 29
(Formulation 26, Example 32)

| | | Weight |
|---|---|---|
| | Amount | Percentage |
| Ingredient | (g) | (%) |
| Part I | | |
| Propylene Carbonate (liquid) | 8.00 | 8.92 |
| Dipropylene Glycol (liquid) | 8.00 | 8.92 |
| Dibenzilidine Sorbital (solid) | 3.70 | 4.12 |
| Subtotal | 19.70 | 21.96 |
| Part II | | |
| Steareth-20 | 12.00 | 13.38 |
| Steareth-2 | 6.00 | 6.69 |
| C₁₂-C₁₅ Alkyl Benzoate | 6.00 | 6.69 |
| Propylene Glycol | 44.00 | 49.05 |
| Silsoft 305 | 2.00 | 2.23 |
| TOTAL | 89.70 | 100.00 |

Formulations 25 and 26 were given to a healthy female volunteer in a blind study to test on her underarms during normal daily activities, including strenuous activity. The participant was unaware of the exact formulation and if, in fact, they were different. One formulation was applied always to the right underarm (Formulation 25, Example 31) and the other (Formulation 26, Example 32) to the left underarm. The participant was asked to rate the relative odor of each underarm as well as the clothing touching that underarm at the end of each day. The participant consistently noted for seven consecutive days that the right underarm and clothing removed from the right underarm had less odor than the left underarm and clothing removed from the left underarm.

Both the deodorants (Example 31 and Example 32) formed a clear stick and were fragrance-free. The stick in Example 31 (Formulation 25) prevented malodor. The stick in Example 32 (Formulation 26, which did not contain CHDM-D or CHDM-D90) did not prevent underarm odor.

### Example 33

### Table 30, Formulation 27, Clear Antiperspirant Stick with 1,4-CHDM

An antiperspirant stick was prepared by first adding the liquid ingredients of Part I (listed in Table 30, Formulation 27) together and heated to 95° C. Dibenzilidene sorbital was then added until it dissolved. The solution was kept at 85° C.

Meanwhile, Steareth-2 and Steareth-20 of Part II were melted at 85° C. in the presence of C₁₂-C₁₅ alkyl benzoate, Silsoft 305, and propylene glycol to form a Part II solution.

The Part II solution was then added to the Part I solution and was heated while stirring with a magnetic stir bar to 90-95° C. At 90-95° C., 22.70 g of the mixture was removed and added to a second container. 4.32 g of aluminum chlorohydrate was stirred in to the 22.70 g of the mixture. It was cooled to 82° C. and poured into deodorant containers.

TABLE 30
(Formulation 27, Example 33)

| | | Weight |
|---|---|---|
| | Amount | Percentage |
| Ingredient | (g) | (%) |
| Part I | | |
| CHDM-D (liquid) | 4.00 | 3.75 |
| Propylene Carbonate (liquid) | 8.00 | 7.49 |
| Dipropylene Glycol (liquid) | 8.00 | 7.49 |
| Dibenzilidine Sorbital (solid) | 3.70 | 3.47 |
| Part II | | |
| Steareth-20 | 12.00 | 11.24 |
| Steareth-2 | 6.00 | 5.62 |
| C₁₂-C₁₅ Alkyl Benzoate | 6.00 | 5.62 |
| Silsoft 305 | 2.00 | 1.87 |
| Propylene Glycol | 40.00 | 37.46 |
| SUBTOTAL | 89.70 | 84.01 |
| Part I + Part II, (combined) | 22.70 | 84.01 |
| Aluminum Chlorohydrate | 4.32 | 15.99 |
| Total | 27.02 | 100.00 |

### Examples 34

### Microbiological Challenge Testing

The microorganisms in the table below were used in challenge tests. They were either ATCC (American Type Culture Collection) or “wild type” as indicated. Wild type organisms were problematic organisms previously isolated from chemical products.

| | |
|---|---|
| Candida albicans | Wild type |
| Aspergillus niger | Wild type |
| Burkholderia cepacia | Wild type |
| Staphylococcus aureus | ATCC 25923 |
| Pseudomonas aeruginosa | Wild type |
| Escherichia coli | ATCC 25922 |
| Bacillus subtilis | Wild type |
| Proteus vulgaris | Wild type |
| Aeromonas sp. | Wild type |
| Staphylococcus epidermidis | ATCC 12228 |
| Streptococcus mutans | ATCC 35668 |

All of the microorganisms were grown in Tryptose Soy Broth (TSB), DIFCO™ available from Becton, Dickinson and Company, containing 1% dextrose. Candida albicans and Aspergillus niger, were incubated at 22° C.+/−2° C. for at least 96 hours. All bacteria were incubated at 35° C.+/−2° C. in a humidified incubator for at least 96 hours.

Candida albicans and Aspergillus niger were also grown on Sabouraud Dextrose Agar (SABD) at 22° C. for 7 to 14 days or until full sporulation was achieved.

### Determining the Amount of Challenge Inocula

The following procedure was followed to determine the amount of each challenge material (inoculum broth) needed to produce a 10⁸ CFU/mL challenge, which is equivalent to a final test-sample microbial concentration of 10⁵ to 10⁶ CFU/mL (CFU=colony forming units).

Using a sterile pipette, 1 mL of the growth from each TSB culture was transferred into tubes of 9 mL buffered water (pH 7.2 phosphate buffer) and mixed thoroughly. This was repeated to make serial 1:10 dilutions. Then, 0.1 mL of each sample and dilution was inoculated onto agar plates to produce the equivalent of a further 1:10 dilution. (C. albicans and A. niger were inoculated onto SABD and bacteria were inoculated onto Plate Count Agar (PCA), DIFCO™ available from Becton, Dickinson and Company.) (The normal volume plated for most samples was 0.1 mL. Some samples that could be very low in micro-organisms (high dilutions) were plated using 0.5 mL of inoculum, in order to produce reproducible counts.) The samples were distributed on the plates using the spread-plate technique. The spread-plate technique is carried out by spreading the sample over the entire plate surface using a sterile spreading rod while rotating the plate with a rotary auto-plater. After the inoculum was absorbed completely by the agar, each plate was inverted and incubated (fungi at 22° C.+/−2° C. and bacteria at 35° C.+/−2° C.).

After incubation for at least 48 hours, colonies that had developed on the agar plates were counted (CFU) and recorded with the corresponding dilution. If counting had to be delayed temporarily, plates were refrigerated, preferably no more than 24 hours, until they could be counted. The number of CFU/mL was determined by multiplying the plate count by the dilution factor of the plate counted.

Turbidity in Nepholemetric Turbidity Units (NTU) was measured for each serial dilution using the HF-Micro 100 Model Turbidimeter. For each microorganism, the plate counts were compared to the turbidity readings. For Candida albicans and all bacteria, the 1:10 dilution having a turbidity reading of 34 to 38 NTU achieved a final test-sample concentration of 10⁵ to 10⁶ CFU/mL. For Aspergillus niger, the 1:10 dilution having a turbidity reading of 25 to 29 NTU achieved the final test-sample concentration of 10⁵ to 10⁶ CFU/mL.

Harvesting Candida albicans Cultures

On the day of challenge, the Candida albicans inoculum broth was poured through nonabsorbent sterile gauze and centrifuged. The pellicle was then diluted with buffered water (pH 7.2 phosphate buffer) until the desired turbidity was reached. Using a hemocytometer, a determination was made whether the challenge contained the desired concentration. Dilutions were made through 1.0×10⁸ and three SABD spread plates were inoculated with 0.1 mL of each dilution. The plates were incubated for at least 48 hours and challenge counts were confirmed.

Harvesting Aspergillus niger Cultures and Dislodging Spores

Aspergillus niger cultures were harvested and spores dislodged from the SABD on which they were grown by rubbing the growth gently with a sterile inoculating loop. The spores were then mixed into the broth culture that had been incubated with a sterile magnetic stir bar to reduce pellicle formation. The spore-culture mixture was filtered repeatedly through sterile, nonabsorbent cotton and harvested repeatedly, adjusting vegetative cells and spores to a level of 1.0×10⁸. A hemocytometer was used to verify the final challenge concentration.

### Preparation of Test Substrates

Control substrates (“broth alone”) were prepared for each microorganism separately in triplicate by adding 13.5 mL of Buffered Peptone Water broth (BPW) containing 1% dextrose to each 20-mL glass tube, then adding 1.5 mL challenge material to produce a final concentration at time zero of 10⁵ to 10⁶ CFU/mL and a total volume of 15 mL.

Test sample substrates were prepared containing each test material listed below at the concentrations shown for challenge testing with each microorganism separately. Sample substrates were prepared in triplicate, except the substrates containing butoxyethanol which were prepared in duplicate. Substrates were prepared by adding BPW containing 1% dextrose to each 20-mL glass tube, then adding the test material in the amount appropriate to achieve the desired weight/volume percent and to achieve a total volume of BPW plus dextrose plus test material of 13.5 mL. Then 1.5 mL challenge material was added to produce a final concentration at time zero of 10⁵ to 10⁶ CFU/mL and a total test sample substrate volume of 15 mL.

| | Test Concentration, % (wt/vol) | | | |
|---|---|---|---|---|
| Test Material | 0.5 | 1.25 | 2.5 | 5.0 |
| 1,2-cyclohexanedimethanol | x | x | x | x |
| 1,4-cyclohexanedimethanol | x | x | x | x |
| 2,2,4,4,-tetramethyl-1,3-cyclobutanediol | x | x | x | x |
| (TMCBD) | | | | |
| 1,3-cyclohexanedimethanol | x | x | x | x |
| 2-butoxyethanol (EB) | NT | NT | x | x |
| propylene glycol (PG) | x | x | x | x |
| Na₂EDTA | x | x | x | x |

NT = Not tested.

### Incubation and Microbiological Testing

After mixing, all challenged substrates were incubated at 35° C.±2° C. for 14 days and ambient room temperature after 14 days.

Subcultures were performed at 3, 14, and 30 days as follows:

A 0.1-mL aliquot was removed from each challenged substrate. The turbidity of the sample was determined and if needed to produce readable plate counts (see “Plate Counts” below), the aliquot was diluted with buffered water (pH 7.2 phosphate buffer). Candida albicans and Aspergillus niger were subcultured onto SABD and grown at 22° C.+/−2° C. The bacteria were subcultured onto PCA and incubated at 35° C.+/−2° C. in a humidified incubator. Negative results were not reported before 96 hours incubation and counts were performed after no less than 48 hours incubation.

The identity of the microorganisms was confirmed by Gram Stain or Cotton Blue staining whenever contamination was suspected. INT Respiratory Dye (p-iodonitrotetrazolium violet available from Sigma Chemical Company), Gram Stain, and the ATP (Adenosine Triphosphate) test were used whenever negative results were questioned because of cloudiness in the tube.

### Plate Counts

For diluted samples, plates producing 22 to 220 counts per plate were counted and the count was multiplied by the dilution factor.

### Interpretation of Data

| Grade | Definition of Grade for C. albicans and All Bacteria |
|---|---|
| 0 | No colonies detected for 0.1 mL sample = No Growth |
| 1 | 0 to 51 colonies counted (thus 10 to 510 CFU/mL based on |
| | 0.1-mL sample) |
| 2 | 52 to 100 colonies counted (thus 520 to 1000 CFU/mL) |
| 3 | 100 to 1000 colonies (thus 1000 to 10,000 CFU/mL) |
| 4 | 1000 to 10,000 colonies countable (thus 10,000 CFU/mL to |
| | 100,000 CFU/mL) |
| 5 | More than 10,000 colonies estimated (thus more than |
| | 100,000 CFU/mL) |

| Grade | Definition of Grade for A. niger |
|---|---|
| 0 | No growth demonstrated from 0.1 mL subculture sample |
| 1 | Countable (1 to 10 colonies) |
| 2 | Countable (10 to 100 colonies) |
| 3 | Individual colonies not countable; over 75% of plate covered |
| | with growth |
| 4 | Plate is not countable; one continuous mat of fungi |
| 5 | Obvious extreme growth (even macroscopically) in tube |

The results are shown in Tables 31-35 below. The concentrations shown are expressed as weight/volume percent. The test procedure for M. furfur, M. canis, and T. rubrum (results included in Tables 34 and 35) is given in Example 10.

TABLE 31

| | A. niger | | | C. albicans | | | B. cepacia | | |
|---|---|---|---|---|---|---|---|---|---|
| | 3 day | 13 day | 30 day | 3 day | 14 day | 30 day | 3 day | 14 day | 30 day |
| Broth | 5 | 5 | 5 | 5 | 5 | 5 | 5 | 5 | 5 |
| Alone | | | | | | | | | |
| 0.5% | | | | | | | | | |
| 1,2-CHDM | 4.7 | 4 | 4 | 4 | 4 | 4 | 4 | 4 | 4 |
| 1,4-CHDM | 4 | 4 | 4 | 3 | 4 | 4 | 4 | 4 | 4 |
| TMCBD | 5 | 5 | 5 | 4 | 4 | 4 | 4 | 4 | 4 |
| 1,3-CHDM | 5 | 5 | 5 | 4 | 4 | 4 | 5 | 5 | 5 |
| PG | 5 | 5 | 5 | 5 | 5 | 5 | 5 | 5 | 5 |
| EDTA | 4 | 4 | 4 | 4 | 5 | 5 | 4 | 4 | 4 |
| 1.25% | | | | | | | | | |
| 1,2-CHDM | 3.3 | 3.3 | 4 | 4 | 4 | 4 | 1 | 0 | 0 |
| 1,4-CHDM | 4 | 4 | 4 | 1 | 3 | 4 | 4 | 2 | 4 |
| TMCBD | 4 | 4 | 4 | 3 | 4 | 4 | 3 | 2 | 4 |
| 1,3-CHDM | 5 | 4 | 4 | 4 | 4 | 4 | 4.7 | 4.7 | 4.7 |
| PG | 5 | 5 | 5 | 5 | 5 | 5 | 5 | 5 | 5 |
| EDTA | 2 | 1 | 3 | 4 | 4 | 4 | 3 | 2 | 2 |
| 2.5% | | | | | | | | | |
| 1,2-CHDM | 2 | 0 | 0 | 0 | 0 | 0 | 0 | 0 | 0 |
| 1,4-CHDM | 2 | 1 | 2.3 | 0 | 2 | 1 | 0 | 0 | 0 |
| TMCBD | 3 | 2 | 3 | 3 | 2 | 2 | 1 | 0 | 0 |
| 1,3-CHDM | 4 | 4 | 4 | 4 | 4 | 4 | 4 | 4 | 4 |
| EB | 4 | 4 | 4 | 4 | 4 | 4 | 4 | 4 | 4 |
| PG | 4 | 5 | 5 | 4 | 4 | 4 | 5 | 5 | 5 |
| EDTA | 0 | 0 | 0 | 2 | 2 | 2 | 1 | 0 | 0 |
| 5.0% | | | | | | | | | |
| 1,2-CHDM | 0 | 0 | 0 | 0 | 0 | 0 | 0 | 0 | 0 |
| 1,4-CHDM | 0 | 0 | 0 | 0 | 0 | 0 | 0 | 0 | 0 |
| TMCBD | 2 | 0 | 0 | 2 | 0 | 0 | 1 | 0 | 0 |
| 1,3-CHDM | 2 | 2 | 4 | 0 | 2 | 3 | 3 | 1 | 3.3 |
| EB | 2.5 | 0 | 0 | 2 | 0 | 0 | 0 | 0 | 0 |
| PG | 4 | 4 | 4 | 3 | 4 | 4 | 4 | 4 | 4 |
| EDTA | 0 | 0 | 0 | 1 | 0 | 0 | 0 | 0 | 0 |

TABLE 32

| | S. aureus | | | P. aeruginosa | | | E. Coli | | |
|---|---|---|---|---|---|---|---|---|---|
| | 3 day | 14 day | 30 day | 3 day | 13 day | 30 day | 3 day | 14 day | 30 day |
| Broth | 5 | 5 | 5 | 5 | 5 | 5 | 5 | 5 | 5 |
| Alone | | | | | | | | | |
| 0.5% | 4 | 4 | 4 | 4 | 4 | 4 | 5 | 5 | 5 |
| 1,2-CHDM | 4 | 4 | 4 | 4 | 4 | 4 | 5 | 4 | 4 |
| 1,4-CHDM | 4 | 4 | 4 | 4 | 4 | 4 | 5 | 4 | 4 |
| TMCBD | 4 | 4 | 4 | 5 | 5 | 5 | 5 | 5 | 5 |
| 1,3-CHDM | 5 | 5 | 5 | 5 | 5 | 5 | 5 | 5 | 5 |
| PG | 5 | 4 | 4 | 4 | 4 | 5 | 5 | 5 | 5 |
| EDTA | 4 | 4 | 4 | 4 | 4 | 4 | 5 | 5 | 5 |
| 1.25% | | | | | | | | | |
| 1,2-CHDM | 3.3 | 2.7 | 4 | 2.7 | 1 | 3 | 4 | 2 | 3 |
| 1,4-CHDM | 3 | 2.7 | 4 | 4 | 3 | 4 | 4 | 4 | 4 |
| TMCBD | 4 | 4 | 4 | 4 | 4 | 4 | 4 | 4 | 4 |
| 1,3-CHDM | 4 | 4 | 4 | 4 | 4 | 4 | 5 | 4 | 4 |
| PG | 4 | 4 | 4 | 4 | 4 | 4 | 5 | 4 | 4 |
| EDTA | 2.7 | 2 | 2 | 4 | 4 | 4 | 4 | 4 | 4 |
| 2.5% | | | | | | | | | |
| 1,2-CHDM | 0 | 0 | 0 | 1 | 0 | 0 | 2 | 0 | 0 |
| 1,4-CHDM | 1.7 | 1 | 0 | 3 | 2 | 0 | 3 | 2 | 0 |
| TMCBD | 2 | 1 | 1 | 4 | 3 | 4 | 4 | 4 | 4 |
| 1,3-CHDM | 4 | 4 | 4 | 4 | 4 | 4 | 5 | 4 | 4 |
| EB | 4 | 4 | 4 | 4 | 4 | 4 | 4 | 4 | 4 |
| PG | 4 | 4 | 4 | 4 | 4 | 4 | 5 | 4 | 4 |
| EDTA | 1 | 0.3 | 0.3 | 4 | 3 | 4 | 0 | 0 | 0 |
| 5.0% | | | | | | | | | |
| 1,2-CHDM | 0 | 0 | 0 | 0 | 0 | 0 | 0 | 0 | 0 |
| 1,4-CHDM | 0 | 0 | 0 | 0 | 0 | 0 | 0 | 0 | 0 |
| TMCBD | 0 | 0 | 0 | 3 | 1 | 0 | 0 | 0 | 0 |
| 1,3-CHDM | 4 | 3 | 4 | 4 | 4 | 4 | 4 | 3 | 4 |
| EB | 2 | 0 | 0 | 0 | 0 | 0 | 1 | 0 | 0 |
| PG | 4 | 4 | 4 | 4 | 4 | 4 | 4 | 4 | 4 |
| EDTA | 0 | 0 | 0 | 0 | 0 | 0 | 0 | 0 | 0 |

TABLE 33

| | B. subtillis | | | P. vulgaris | | | Aeromonas sp | | |
|---|---|---|---|---|---|---|---|---|---|
| | 3 day | 14 day | 30 day | 3 day | 13 day | 30 day | 3 day | 14 day | 30 day |
| Broth | 5 | 5 | 5 | 5 | 5 | 5 | 5 | 5 | 5 |
| Alone | | | | | | | | | |
| 0.5% | | | | | | | | | |
| 1,2-CHDM | 4 | 3 | 4 | 4 | 4 | 4 | 5 | 4 | 4 |
| 1,4-CHDM | 4 | 4 | 4 | 4 | 4 | 4 | 5 | 4 | 4 |
| TMCBD | 4 | 4 | 4 | 4 | 4 | 4 | 5 | 4 | 4 |
| 1,3-CHDM | 4 | 4 | 4 | 5 | 5 | 5 | 5 | 4 | 4 |
| PG | 5 | 5 | 5 | 4 | 4 | 4 | 5 | 5 | 5 |
| EDTA | 4 | 4 | 4 | 5 | 4 | 4 | 5 | 4 | 4 |
| 1.25% | | | | | | | | | |
| 1,2-CHDM | 2 | 1 | 1 | 2.3 | 1 | 0 | 3 | 2 | 1 |
| 1,4-CHDM | 4 | 4 | 4 | 3 | 2 | 0 | 4 | 3 | 2 |
| TMCBD | 4 | 4 | 4 | 3 | 2 | 0 | 4 | 4 | 4 |
| 1,3-CHDM | 4 | 4 | 4 | 5 | 4 | 4 | 4 | 4 | 4 |
| PG | 5 | 4 | 4 | 4 | 4 | 4 | 5 | 5 | 5 |
| EDTA | 2 | 2 | 2 | 3 | 1 | 3 | 4 | 4 | 4 |
| 2.5% | | | | | | | | | |
| 1,2-CHDM | 0 | 0 | 0 | 1 | 0 | 0 | 0 | 1 | 0 |
| 1,4-CHDM | 3 | 2 | 1 | 1 | 0 | 0 | 3 | 1 | 0 |
| TMCBD | 4 | 4 | 4 | 4 | 1 | 0 | 4 | 2 | 3 |
| 1,3-CHDM | 4 | 4 | 4 | 4 | 4 | 4 | 4 | 4 | 4 |
| EB | 4 | 4 | 4 | 4 | 4 | 4 | 4 | 4 | 4 |
| PG | 5 | 4 | 4 | 4 | 4 | 4 | 5 | 5 | 5 |
| EDTA | 0 | 0 | 0 | 4 | 4 | 4 | 3 | 2 | 1 |
| 5.0% | | | | | | | | | |
| 1,2-CHDM | 0 | 0 | 0 | 0 | 0 | 0 | 0 | 0 | 0 |
| 1,4-CHDM | 1 | 0 | 0 | 0 | 0 | 0 | 0 | 0 | 0 |
| TMCBD | 4 | 2 | 0 | 0 | 0 | 0 | 2 | 0 | 0 |
| 1,3-CHDM | 4 | 4 | 4 | 4 | 4 | 4 | 4 | 4 | 4 |
| EB | 1 | 0 | 0 | 0 | 0 | 0 | 1 | 0 | 0 |
| PG | 5 | 4 | 4 | 4 | 4 | 4 | 5 | 4 | 4 |
| EDTA | 0 | 0 | 0 | 4 | 4 | 4 | 1 | 0 | 0 |

TABLE 34

| | S. epidermidis | | | S. mutans | | | M. furfur | | |
|---|---|---|---|---|---|---|---|---|---|
| | 3 day | 13 day | 30 day | 3 day | 13 day | 30 day | 3 day | 14 day | 30 day |
| Broth | 5 | 5 | 5 | 5 | 5 | 5 | 0 | 3 | 3 |
| Alone | | | | | | | | | |
| 0.5% | | | | | | | | | |
| 1,2-CHDM | 4 | 3 | 4 | 4 | 4 | 4 | 0 | 0 | 0 |
| 1,4-CHDM | 4 | 4 | 4 | 4 | 4 | 4 | 0 | 1 | 2 |
| TMCBD | 4 | 4 | 4 | 4 | 4 | 4 | 0 | 1 | 2 |
| 1,3-CHDM | 4 | 4 | 4 | 5 | 4 | 4 | 0 | 1 | 3 |
| PG | 5 | 5 | 5 | 5 | 5 | 5 | 0 | 3 | 3 |
| EDTA | 4 | 3 | 4 | 4 | 4 | 4 | 0 | 0 | 0 |
| 1.25% | | | | | | | | | |
| 1,2-CHDM | 2.3 | 1 | 3 | 2 | 0 | 0 | 0 | 0 | 0 |
| 1,4-CHDM | 2 | 0.7 | 2 | 4 | 2 | 2 | 0 | 0 | 0 |
| TMCBD | 2 | 3 | 4 | 4 | 4 | 4 | 0 | 1 | 2 |
| 1,3-CHDM | 4 | 3.7 | 4 | 4 | 4 | 4 | 0 | 2 | 3 |
| PG | 4 | 4 | 4 | 4 | 4 | 4 | 0 | 3 | 3 |
| EDTA | 2 | 1 | 0 | 2 | 2 | 2 | 0 | 0 | 0 |
| 2.5% | | | | | | | | | |
| 1,2-CHDM | 0 | 0 | 0 | 1 | 0 | 0 | 0 | 0 | 0 |
| 1,4-CHDM | 0.7 | 0 | 0 | 2 | 0 | 0 | 0 | 0 | 0 |
| TMCBD | 2 | 2 | 2 | 3 | 1 | 0 | 0 | 0 | 0 |
| 1,3-CHDM | 4 | 3 | 4 | 4 | 4 | 4 | 0 | 0 | 0 |
| EB | 4 | 4 | 4 | 4 | 4 | 4 | NT | NT | NT |
| PG | 4 | 4 | 4 | 4 | 4 | 4 | 0 | 3 | 3 |
| EDTA | 0 | 0 | 0 | 0 | 0 | 0 | 0 | 0 | 0 |
| 5.0% | | | | | | | | | |
| 1,2-CHDM | 0 | 0 | 0 | 0 | 0 | 0 | 0 | 0 | 0 |
| 1,4-CHDM | 0 | 0 | 0 | 0 | 0 | 0 | 0 | 0 | 0 |
| TMCBD | 1 | 0 | 0 | 0 | 0 | 0 | 0 | 0 | 0 |
| 1,3-CHDM | 2 | 1 | 3 | 2 | 0 | 0 | 0 | 2 | 3 |
| EB | 0 | 0 | 0 | 0 | 0 | 0 | NT | NT | NT |
| PG | 4 | 4 | 4 | 4 | 4 | 4 | 0 | 0 | 3 |
| EDTA | 0 | 0 | 0 | 0 | 0 | 0 | 0 | 0 | 0 |

TABLE 35

| | M. canis | | | T. rubrum | | |
|---|---|---|---|---|---|---|
| | 3 day | 14 day | 30 day | 3 day | 14 day | 30 day |
| Broth Alone | 2 | 1 | 1 | 1 | 0 | 3 |
| 0.5% | | | | | | |
| 1,2-CHDM | 2 | 1 | 1 | 0 | 0 | 0 |
| 1,4-CHDM | 2 | 1 | 1 | 1 | 1 | 1 |
| TMCBD | 2 | 1 | 1 | 1 | 1 | 0 |
| 1,3-CHDM | 2 | 1 | 1 | 0 | 2 | 1 |
| PG | 2 | 1 | 2 | 0 | 1 | 1 |
| EDTA | 2 | 1 | 0 | 1 | 0 | 0 |
| 1.25% | | | | | | |
| 1,2-CHDM | 1 | 0 | 0 | 0 | 0 | 0 |
| 1,4-CHDM | 2 | 1 | 1 | 0 | 0 | 0 |
| TMCBD | 2 | 1 | 0 | 0 | 0 | 1 |
| 1,3-CHDM | 2 | 1 | 1 | 1 | 0 | 0 |
| PG | 2 | 1 | 1 | 1 | 0 | 1 |
| EDTA | 2 | 1 | 0 | 0 | 0 | 0 |
| 2.5% | | | | | | |
| 1,2-CHDM | 0 | 0 | 0 | 1 | 0 | 0 |
| 1,4-CHDM | 2 | 0 | 0 | 0 | 0 | 0 |
| TMCBD | 2 | 1 | 1 | 0 | 0 | 0 |
| 1,3-CHDM | 0 | 0 | 0 | 0 | 0 | 0 |
| EB | NT | NT | NT | NT | NT | NT |
| PG | 1 | 1 | 2 | 1 | 0 | 1 |
| EDTA | 2 | 1 | 0 | 1 | 0 | 0 |
| 5.0% | | | | | | |
| 1,2-CHDM | 0 | 0 | 0 | 0 | 0 | 0 |
| 1,4-CHDM | 0 | 0 | 0 | 1 | 0 | 0 |
| TMCBD | 2 | 1 | 1 | 0 | 0 | 0 |
| 1,3-CHDM | 2 | 1 | 1 | 2 | 0 | 0 |
| EB | NT | NT | NT | NT | NT | NT |
| PG | 1 | 1 | 2 | 1 | 0 | 1 |
| EDTA | 2 | 0 | 0 | 1 | 0 | 0 |

Comparing 1,2-CHDM, 1,4-CHDM, and TMCBD to 1,3-CHDM, 2-butoxyethanol, and propylene glycol in Tables 31-35 above, differences of 2-log or greater for the same organism and time period were considered unexpected.

At 0.5% glycol concentration, the differences and effectiveness were not that great.

At 1.25% glycol concentration, results for some of the organisms were unexpected with 1,2-CHDM having the most unexpected results relative to 1,3-CHDM. EB solvent (2-butoxyethanol) was not tested at 1.25%.

At 2.5% glycol concentration, with the exception of M. canis and T. rubrum, all results for 1,2-CHDM and most results for 1,4-CHDM were unexpected in light of results for 1,3-CHDM and for EB solvent. The microbe counts for 1,3-CHDM and EB solvent were 4-log for almost all organisms tested, indicating an insignificant preservative effect.

At 5.0% glycol concentration, with the exception of M. canis and T. rubrum, all results for 1,2-CHDM and 1,4-CHDM, and most results for TMCBD were unexpected in light of results for propylene glycol and most of the results for 1,3-CHDM.

Fungi M. canis and T. rubrum were particularly difficult to grow, and therefore showed significantly fewer unexpected results.

The invention has been described in detail with particular reference to preferred embodiments thereof, but it will be understood that variations and modifications can be effected within the spirit and scope of the invention.

We claim:

1. A method for reducing or inhibiting microbial growth in an aqueous composition comprising:
adding an antimicrobial agent selected from the group consisting of 1,2-cyclohexanedimethanol, 1,4-cyclohexanedimethanol, and 2,2,4,4-tetramethyl-1,3-cyclobutanediol to the composition.

2. The method according to claim 1, wherein the antimicrobial agent is added in an amount of about 1 to 5 weight percent, based on the total weight of the composition.

3. The method according to claim 1, wherein the antimicrobial agent is added in an amount of about 1 to 3 weight percent, based on the total weight of the composition.

4. The method according to claim 1, wherein the antimicrobial agent is 1,2-cyclohexanedimethanol.

5. The method according to claim 1, wherein the antimicrobial agent is 1,4-cyclohexanedimethanol.

6. The method according to claim 1, wherein the antimicrobial agent is 2,2,4,4-tetramethyl-1,3-cyclobutanediol.

7. The method according to claim 1, where the microbial growth is a biofilm.

8. The method according to claim 1, wherein the antimicrobial agent is added to the aqueous composition by contacting the aqueous composition with a solvent that is immiscible with water and that comprises the antimicrobial agent.

9. The method according to claim 1, wherein the aqueous composition comprises an organic compound selected from hydrocarbons, triglycerides, fatty acids, fatty acid alkyl esters, fatty alcohols, polyglycol ethers, alkyl glycol ethers, alkyl glycol esters, alkyl glycol ether esters, alkyl amines, alkyl amides, or mixtures thereof.

10. The method according to claim 9, wherein the organic compound and water in the aqueous composition are miscible.

11. The method according to claim 9, wherein the organic compound and water in the aqueous composition are in separate liquid phases, and the method reduces or inhibits microbial growth at the interface between the organic phase and the aqueous phase.

12. The method according to claim 9 or 11, wherein the organic compound is diesel, biodiesel, a mixture of diesel and biodiesel, aviation fuel, hydraulic oil, lubrication oil, vegetable oil, crude oil, transmission fluid, heating oil, or kerosene.

13. A method for providing residual antimicrobial activity to a surface, said method comprising:
topically applying a composition comprising about 1 to 5 weight percent of an antimicrobial agent selected from the group consisting of 1,2-cyclohexanedimethanol, 1,4-cyclohexanedimethanol, and 2,2,4,4-tetramethyl-1,3-cyclobutanediol to the surface; and
optionally removing any excess amounts of the product from the surface.

14. The method according to claim 13, wherein the surface has a biofilm before applying the product.

15. A method for preventing or treating a bacterial or fungal infection on a mammalian surface, said method comprising:
topically applying a composition comprising about 1 to 5 weight percent of an antimicrobial agent selected from the group consisting of 1,2-cyclohexanedimethanol, 1,4-cyclohexanedimethanol, and 2,2,4,4-tetramethyl-1,3-cyclobutanediol to the mammalian surface; and
optionally removing any excess amounts of the product from the mammalian surface.

16. A method for preventing or reducing odor from the presence of bacteria or fungi on a mammalian surface, said method comprising:
topically applying a composition comprising about 1 to 5 weight percent of an antimicrobial agent selected from the group consisting of 1,2-cyclohexanedimethanol, 1,4-cyclohexanedimethanol, and 2,2,4,4-tetramethyl-1,3-cyclobutanediol to the mammalian surface; and
optionally removing any excess amounts of the product from the mammalian surface.

17. A method for providing antimicrobial activity to a film, fiber, molded or extruded article, or composite material made of fibers, polymers, adhesives, and/or gypsum; said method comprising:
incorporating an antimicrobial agent selected from the group consisting of 1,2-cyclohexanedimethanol, 1,4-cyclohexanedimethanol, and 2,2,4,4-tetramethyl-1,3-cyclobutanediol into the film, fiber, molded or extruded article, or composite material during its manufacturing process.

18. The method according to claim 17, which prevents a biofilm from forming on a surface of the film, fiber, molded or extruded article, or composite material.

19. The method according to claim 17, wherein the antimicrobial agent is incorporated in an amount of about 1 to 5 weight percent, based on the total weight of the film, fiber, molded or extruded article, or composite material.

20. The method according to claim 17, wherein the antimicrobial agent is incorporated in an amount of about 1 to 3 weight percent, based on the total weight of the film, fiber, molded or extruded article, or composite material.

21. The method according to claim 17, wherein the antimicrobial agent is 1,2-cyclohexanedimethanol.

22. The method according to claim 17, wherein the antimicrobial agent is 1,4-cyclohexanedimethanol.

23. The method according to claim 17, wherein the antimicrobial agent is 2,2,4,4-tetramethyl-1,3-cyclobutanediol.
